(19) United States
(12) Patent Application Publication
(10) Pub. No.: US 2008/0124736 A1
(43) Pub. Date: May 29, 2008

(54) Detection of virulence markers of staphylococci

(75) Inventors: Mark Fiandaca, Princeton, MA (US); Anne Rasmussen, Taastrup (DK); Henrik Stender, Gentofte (DK)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: AdvanDx, Inc., Woburn, MA (US)

(21) Appl. No.: 11/975,685

(22) Filed: Oct. 19, 2007

Related U.S. Application Data
(63) Continuation-in-part of application No. PCT/US2006/014971, filed on Apr. 21, 2006.
(60) Provisional application No. 60/735,061, filed on Nov. 8, 2005.
(60) Provisional application No. 60/673,900, filed on Apr. 21, 2005.

(51) Int. Cl. C12Q 1/68; C40B 40/06; C07H 21/04

(52) U.S. Cl. 435/6; 536/24.32; 506/16

(57) ABSTRACT

Methods, kits and compositions are provided for detection of virulence markers in pathogenic organisms. More specifically, methods, kits, arrays, and compositions are described which enable detection of marker genes useful in classifying Staphylococcus species, particularly Staphylococcus aureus. Compositions of probes targeted against virulence genes encoding Panton-Valentine leukocidin (PVL), Toxic shock syndrome toxin-1 (TSST-1) and mupirocin resistance (encoded by ileS-2) are described.

### RELATED APPLICATIONS

This application is a continuation-in-part of International Application No. PCT/US2006/014971, which designated the United States and was filed on Apr. 21, 2006, published in English, which claims the benefit of U.S. Provisional Application No. 60/673,900, filed on Apr. 21, 2005, and U.S. Provisional Application No. 60/735,061, filed on Nov. 8, 2005. The entire teachings of the above applications are incorporated herein by reference.

### BACKGROUND OF THE INVENTION

Release of TSST-1 into the bloodstream is closely associated with toxic shock syndrome (TSS) which is characterized by high fever, erythematous rash formation, hypotension and major oxygen involvement. Without appropriate therapy, TSS may lead to multi-organ failure and death. TSST-1 is often associated with cases of menstrual TSS (tampon use) as well as TSS cases which are related to localized infections, surgical complications, insect bites and cosmetic surgery (Dinges et al. Clin Microbiol Rev. 2000, 13(1), 16-34; Llewelyn & Cohen, Lancet Infect Dis. 2002, 2(3): 156-62; Deurenberg et al. FEMS Microbiol Lett. 2005, 245(1), 85-9; Holm et al. Aesthetic Plast Surg. 1998 May-Jun;22(3):180-4.). Nonmenstrual TSS has a higher mortality rate than TSS associated with menstruation (Lowy, N Engl J Med. 1998, 339(8), 520-32).

TSST-1 is a potent polyclonal T cell mitogen. TSST-1 activates T cells by cross-bridging the major histocompatibility complex class II molecules of antigen presenting cells with T cell receptors thus inducing non-specific cytokine release and proliferation of lymphocytes (Peterson et al. Infection and Immunity 2005, 73 (4): 2164-2174). By bypassing the normal antigen presentation mechanism, the TSST-1 “superantigen” stimulates a much larger percentage of T cells than a conventional antigen response (MacIsaac et al. MJA 2005 182 (12): 651-652).

In S. aureus the lukF-PV and lukS-PV genes are contiguous and co-transcribed and their secreted gene products work in conjunction to produce the exotoxin, Panton-Valentine leukocidin. An assay designed to test for the presence of either or both of the lukF-PV and lukS-PV genes yielding a positive result, therefore, is an indication that both genes are present.

The PVL exotoxin kills leukocytes by creating pores in the cell membrane. PVL exotoxin has high leukocytolyic activity when tested on human glass-adherent leukocytes, and can produce a localized acute inflammation when tested on rabbit skin (Prevost, Inf and Imm 1995). Also, purified PVL exotoxin tested on rabbit skin has potent and rapid dermonecrotic activity at low concentration.

PVL positive community acquired S. aureus has been implicated in cases of haemorrhagic necrotizing pneumonia in immunocompetent patients (Gillet, Lancet 2002, 359, 753-759). PVL positive strains were shown to produce a more rapid, progressive and ultimately more fatal disease.

Beyond its importance as a virulence marker, PVL is a common hallmark of community acquired strains of methicillin resistant S. aureus (CA-MRSA). The genomes of all MRSA contain a staphylococcal chromosome cassette (SCCmec) carrying the mecA gene for methicillin resistance as well as other virulence and resistance factors. MRSA are classified by their SCCmec types; PVL positive CA-MRSA isolates are frequently of type IV. It has been suggested that the reason the SCCmec type IV is found more commonly in CA-MRSA than other SCCmec types in CA-MRSA is that the relatively small size of cassette IV is less of a burden on the organism. Nosocomial strains of MRSA have larger SCCmec cassettes that include other resistance genes which confer a selective advantage due to the pressure of antibiotic and antimicrobial use in hospitals. CA-MRSA, unlike nosocomial MRSA, tend not to be multidrug resistant and are frequently associated with furunculosis and skin and soft tissue infections (SSTI).

Mupirocin is a naturally occurring polyketide antibiotic targeting isoleucyl-tRNA synthetase. The chemical name of mupirocin is (E)-(2S,3R,4R,5S)-5-[(2S,3S,4S,5S)-2,3-Epoxy-5-hydroxy-4-methylhexyl]tetrahydro-3,4-dihydroxy-b-methyl-2H-pyran-2-crotonic acid, ester with 9-hydroxynonanoic acid. Mupirocin is often used clinically to limit the spread of microorganisms on the skin of patients. Prophylactic use of mupirocin has become a typical pre-operative treatment to prevent the spread of Staphylococcus aureus to surgical sites. Resistance to mupirocin can be acquired through acquisition of the ileS-2 gene which codes for a modified isoleucyl-tRNA synthetase.

Staphylococcus aureus and other staphylococci that carry genes for antibiotic resistance, genes encoding toxins, and genes for other virulence factors are causes of infections that are particularly difficult to treat. Methods and tools to diagnose infections of these organisms and to track their epidemiological patterns are needed.

### SUMMARY OF THE INVENTION

The invention includes probes comprising a nucleobase sequence selected from among SEQ ID NOs 1-23. The invention also comprises probes consisting essentially of any of the nucleobase sequences in Table 1, as well as probes that consist of a nucleobase sequence selected from the group consisting of SEQ ID NOs 1-23.

The invention also includes compositions comprising a probe, the probe comprising a nucleic acid or a DNA mimic wherein the nucleic acid or DNA mimic comprises: a) SEQ ID NO:X; b) a nucleobase sequence having at least 75% sequence identity to SEQ ID NO:X; or c) a contiguous segment of either a) or b) at least 8 nucleobases long. In some cases, the contiguous segments can be at least 20 nucleobases long. SEQ ID NO:X is a nucleobase sequence selected from the group consisting of SEQ ID NOs 1-23. In another embodiment, the nucleic acid or DNA mimic as described above consists of a nucleobase sequence less than or equal to about 100 nucleobases long.

Other compositions of the invention can be described as comprising a probe. The probe can comprise a nucleic acid or a DNA mimic wherein the nucleic acid or DNA mimic consists essentially of: a) SEQ ID NO:X; b) a nucleobase sequence having at least 75% sequence identity to SEQ ID NO:X; c) a contiguous segment of a) at least 8 nucleobases long; or d) a contiguous segment of b) at least 8 nucleobases long. SEQ ID NO:X is a nucleobase sequence selected from the group consisting of SEQ ID NOs 1-23. In certain cases, the contiguous segments can be at least 20 nucleobases long.

Still other compositions of the invention comprise a probe wherein the probe comprises a nucleic acid or a DNA mimic consisting of: a) SEQ ID NO:X; b) a nucleobase sequence having at least 75% sequence identity to SEQ ID NO:X; c) a contiguous segment of a) at least 8 nucleobases long; or d) a contiguous segment of b) at least 8 nucleobases long. SEQ ID NO:X is a nucleobase sequence which is one of SEQ ID NOs 1-23. In some embodiments, the contiguous segments can be at least 20 nucleobases long.

One or more linker moieties, spacer moieties and/or labels can be a part of any of the probes.

Other compositions of the invention allow for the use of certain combinations of probes in assays. The invention is also a composition comprising a) a first probe comprising a nucleobase sequence selected from the group consisting of: i) SEQ ID NO:X; ii) a nucleobase sequence having at least 75% sequence identity to SEQ ID NO:X; iii) a contiguous segment of i) at least 8 nucleobases long; and iv) a contiguous segment of ii) at least 8 nucleobases long; and further comprising b) a second probe comprising a nucleobase sequence selected from the group consisting of: i) SEQ ID NO:X; ii) a nucleobase sequence having at least 75% sequence identity to SEQ ID NO:X; iii) a contiguous segment of i) at least 8 nucleobases long; and iv) a contiguous segment of ii) at least 8 nucleobases long. In this composition, the first and second probes are further described by their SEQ ID NOs wherein X is 1 and Y is 2; X is 1 and Y is 3; X is 1 and Y is 4; X is 1 and Y is 5; X is 2 and Y is 3; X is 2 and Y is 4; X is 2 and Y is 5; X is 3 and Y is 4; X is 3 and Y is 5; or X is 4 and Y is 5. The first and second probes can be described by SEQ ID NOs wherein X is 9 and Y is 10; X is 9 and Y is 11; X is 9 and Y is 15; X is 9 and Y is 16; X is 10 and Y is 11; X is 10 and Y is 15; X is 10 and Y is 16; X is 11 and Y is 15; X is 11 and Y is 16; X is 15 and Y is 16; X is 21 and Y is 22; X is 21 and Y is 23; X is 4 and Y is 22; or X is 4 and Y is 23. The first and second probes can be described by SEQ ID NOs wherein X is 6 and Y is 7; X is 6 and Y is 8; X is 7 and Y is 8; X is 12 and Y is 13; X is 12 and Y is 14; or X is 13 and Y is 14. The first and second probes can be described by SEQ ID NOs wherein X is 17 and Y is 18; or X is 19 and Y is 20.

Probes can be attached to a support in an array, as in a microarray or microtiter plate. Thus, a further aspect of the invention is an array of probes attached to a support, wherein the probes each comprise a nucleic acid or DNA mimic. The probes each can comprise a) SEQ ID NO:X; b) a nucleobase sequence having at least 75% sequence identity to SEQ ID NO:X; c) a contiguous segment of a) at least 8 nucleobases long; or d) a contiguous segment of b) at least 8 nucleobases long. In some embodiments, the contiguous segments can be at least 20 nucleobases long. SEQ ID NO:X can be any of SEQ ID NOs 1-23. In a particular embodiment, the array is as above, and one or more of the probes have sequences selected from each of the groups consisting of: SEQ ID NOs 1-5, 9-11, 15 and 16; SEQ ID NOs 6-8 and 12-14; and SEQ ID NOs 17-20.

Assay methods are also a part of the invention.

In another aspect, the invention is a method of determining the presence or absence of each of a combination of genes in a Staphylococcus strain, using a support. The method involves the following. For each of the genes, fragments of DNA of the Staphylococcus, as from a Staphylococcus strain, are hybridized to one or more capture probes attached to the support. The capture probes have been designed so that they hybridize to a segment of the gene, thereby producing captured DNA comprising a segment of the gene, if fragments of DNA of the Staphylococcus strain comprising a segment of the gene are present among the fragments of the DNA. For each of the genes, one or more detector probes are hybridized to the captured DNA. The detector probes have been designed so that they hybridize to a segment of the captured DNA comprising a segment of the gene, thereby producing hybridized detector probes, if DNA comprising a segment of the gene was captured. The capture probes and detector probes are designed to hybridize to different segments of the target DNA, which, in particular embodiments, can be less than 1000, less than 100, or less than 10 nucleotides apart. For each of the genes, each of the hybridized detector probes is detected or not detected as remaining on the support (that is, tethered to the support through hybridization products), thereby determining the presence or absence, respectively, of each of the genes. In one such method, there is no step in which the DNA is amplified. In another method as described above, hybridization of capture probe to DNA and hybridization of detector probes to DNA can occur at the same time, in the same reaction solution. The above methods can be used to look for various combinations of genes on a support prepared with attached capture probes for that purpose. The combinations include, for example, PVL, tst and ileS-2. Another combination of genes can include: PVL; tst; nuc; mecA; and one or both of vanA and vanB. The method can be carried out with a sample comprising genomic DNA of bacteria, which comprises all DNA in the bacterial cells, chromosomal, or extrachromosomal. However, the method is not limited to the use of genomic DNA of bacteria.

The invention, in another aspect, comprises kits for the detection of genes of Staphylococcus aureus. The genes may also be detected in other species of Staphylococcus.

In one embodiment, a kit of the invention is for detection of the lukS-PV and/or lukF-PV gene of Staphylococcus aureus (test for “PVL”). The kit comprises a set of at least two probes, a) a first probe comprising a nucleobase sequence selected from the group consisting of: i) SEQ ID NO:X; ii) a nucleobase sequence having at least 75% sequence identity to SEQ ID NO:X; iii) a contiguous segment of i) at least 8 nucleobases long; and iv) a contiguous segment of ii) at least 8 nucleobases long; and b) a second probe selected from the group consisting of: i) SEQ ID NO:Y; ii) a nucleobase sequence having at least 75% sequence identity to SEQ ID NO:Y; iii) a contiguous segment of i) at least 8 nucleobases long; and iv) a contiguous segment of ii) at least 8 nucleobases long. Such a kit for detection of PVL can include first and second probes wherein X is 1 and Y is 2; X is 1 and Y is 3; X is 1 and Y is 4; X is 1 and Y is 5; X is 2 and Y is 3; X is 2 and Y is 4; X is 2 and Y is 5; X is 3 and Y is 4; X is 3 and Y is 5; X is 4 and Y is 5; X is 9 and Y is 10; X is 9 and Y is 11; X is 9 and Y is 15; X is 9 and Y is 16; X is 10 and Y is 11; X is 10 and Y is 15; X is 10 and Y is 16; X is 11 and Y is 15; X is 11 and Y is 16; X is 15 and Y is 16; X is 21 and Y is 22; X is 21 and Y is 23; X is 4 and Y is 22; or X is 4 and Y is 23.

In another embodiment, another kit comprises similar components, but includes probes specific for the detection of the tst gene instead of PVL. The kit for tst comprises a set of at least two probes: a) a first probe comprising a nucleobase sequence selected from the group consisting of: i) SEQ ID NO:X; ii) a nucleobase sequence having at least 75% sequence identity to SEQ ID NO:X; iii) a contiguous segment of i) at least 8 nucleobases long; and iv) a contiguous segment of ii) at least 8 nucleobases long; and b) a second probe selected from the group consisting of: i) SEQ ID NO:Y; ii) a nucleobase sequence having at least 75% sequence identity to SEQ ID NO:Y; iii) a contiguous segment of i) at least 8 nucleobases long; and iv) a contiguous segment of ii) at least 8 nucleobases long. Such a kit for detection of tst can include first and second probes wherein X is 6 and Y is 7; X is 6 and Y is 8; X is 7 and Y is 8; X is 12 and Y is 13; X is 12 and Y is 14; or X is 13 and Y is 14.

In a further embodiment, a kit includes probes specific for the detection of the ileS-2 gene. The kit for ileS-2 comprises a set of at least two probes: a) a first probe comprising a nucleobase sequence selected from the group consisting of: i) SEQ ID NO:X; ii) a nucleobase sequence having at least 75% sequence identity to SEQ ID NO:X; iii) a contiguous segment of i) at least 8 nucleobases long; and iv) a contiguous segment of ii) at least 8 nucleobases long; and b) a second probe selected from the group consisting of: i) SEQ ID NO:Y; ii) a nucleobase sequence having at least 75% sequence identity to SEQ ID NO:Y; iii) a contiguous segment of i) at least 8 nucleobases long; and iv) a contiguous segment of ii) at least 8 nucleobases long. Such a kit for detection of ileS-2 can include first and second probes wherein X is 17 and Y is 18; or X is 19 and Y is 20.

### DETAILED DESCRIPTION OF THE INVENTION

The invention is related to the field of probe based nucleic acid sequence detection, analysis and quantification. More specifically, this invention relates to the use of probes for detection of the presence of virulence markers useful in the classification of suspected human pathogens of interest, particularly members of the Staphylococcus genus. In one aspect, the invention allows for typing of Staphylococcus aureus originating from a clinical sample based on the presence or absence of virulence genes encoding toxins such as PVL and TSST-1, or antibiotic resistance genes such as ileS-2. Detection of the presence of a gene targeted by probes of the invention is used to assess the clinical importance of the organism. Scoring of virulence markers is used to direct therapy.

In one aspect, the invention is directed toward detection of the lukF-PV and/or lukS-PV genes, the so called PVL genes of the staphylococci. A test or assay for “PVL” is positive when one or both of lukF-PV and lukS-PV is detected, and negative when neither lukF-PV nor lukS-PV is detected. In a test for PVL, a pair of probes (such as capture and detector probes) can be directed only to one or the other of these genes. Alternatively, because the genes are adjacent, each of a pair of probes can each be directed to a different gene of this pair of genes. Thus, “PVL” when referring to a gene or genes has the meaning lukS-PV or lukS-PV, or both genes, indicating that the tested organism produces PVL exotoxin.

The invention is further directed toward detection of the tst gene which encodes TSST-1.

The invention is further directed toward detection of the ileS-2 gene which encodes a naturally occurring variant of the enzyme isoleucyl-tRNA synthetase. This variant gene is not typically found in staphylococci, but may be acquired. The ileS-2 gene is alternatively named mupA, or simply ileS, depending on the reference. The gene as it is described herein may be found on the Staphylococcus aureus subsp. aureus USA300 plasmid, pUSA03; see NCBI accession number X75439.

Table 1 displays the nucleobase sequences of SEQ ID NOs 1-23.

TABLE 1

| | |
|---|---|
| SEQ ID NO: | Nucleobase Sequence |

| | |
|---|---|
| SEQ ID NO:1 | AACGTATGGCAGAAATATGGATGTTACTCATGCTACTAGA |
| SEQ ID NO:2 | AGTGAACTGGAAAACTCATGAAATTAAAGTGAAAGGACAT |
| SEQ ID NO:3 | GCAACATCAGATTCCGATAAGTTAAAAATTTCTCAGATTT |
| SEQ ID NO:4 | AAAGTTATGATAAAGATACATTAATACTCAAAGCTGCTGG |
| SEQ ID NO:5 | TACAAAGCCAAATCCAAAAGACACTATTAGTTCTCAATTT |
| SEQ ID NO:6 | GCTACAGATTTTACCCCTGTTCCCTTATCATCTAATCA |
| SEQ ID NO:7 | GCATCTACAAACGATAATATAAAGGATTTGCTAGACTGGT |
| SEQ ID NO:8 | TAGTGAAGTTTTAGATAATTCCTTAGGATCTATGCGTATA |
| SEQ ID NO:9 | AAATCTGAGAAATTTTTAACTTATCGGAATCTGATGTTGC |
| SEQ ID NO:10 | CCAGCAGCTTTGAGTATTAATGTATCTTTATCATAACTTT |
| SEQ ID NO:11 | AAATTGAGAACTAATAGTGTCTTTTGGATTTGGCTTTGTA |
| SEQ ID NO:12 | TGATTAGATGATAAGGGAACAGGGGTAAAATCTGTAGC |
| SEQ ID NO:13 | ACCAGTCTAGCAAATCCTTTATATTATCGTTTGTAGATGC |
| SEQ ID NO:14 | TATACGCATAGATCCTAAGGAATTATCTAAAACTTCACTA |
| SEQ ID NO:15 | TCTAGTAGCATGAGTAACATCCATATTTCTGCCATACGTT |
| SEQ ID NO:16 | ATGTCCTTTCACTTTAATTTCATGAGTTTTCCAGTTCACT |
| SEQ ID NO:17 | CTCATTGTTGGAGATGTGGTAATCCTTTGATATATTATG |
| SEQ ID NO:18 | CAATAATAATAATATAGAGTGGTTTCCTTCTCATATTAAG |
| SEQ ID NO:19 | CATAATATATCAAAGGATTACCACATCTCCAACAATGAG |
| SEQ ID NO:20 | CTTAATATGAGAAGGAAACCACTCTATATTATTATTATTG |
| SEQ ID NO:21 | GATAAAGATACATTAATACTCAAAGCTGCTGG |
| SEQ ID NO:22 | CAAAGCCAAATCCAAAAGACACTATTAGTTCTC |
| SEQ ID NO:23 | CAGATTCTAATGACTCAGTAAACGTTGTAG |

The invention includes probes comprising a nucleic acid or a DNA mimic wherein the nucleic acid or DNA mimic comprises, consists essentially of, or consists of a nucleobase sequence selected from the group consisting of: SEQ ID NOs 1-23.

The invention also includes probes comprising a nucleobase sequence which shares at least 75% sequence identity with any of SEQ ID NOs 1-23. Alternatively, the amount of sequence identity can be approximately 80%, 85%, 90% or 95%. In some embodiments the invention includes any of the above probes which is at least 8 nucleobases long, especially in the case of those comprising a DNA mimic. Lengths of some embodiments, especially those comprising nucleic acids, can be at least 20 nucleotides, and can be approximately 30, 40, 50 or more nucleotides. Typical lengths are 38 to 42 nucleobases.

The invention also includes probes that consist of any of the contiguous nucleobase sequences shown in Table 1. Probes consisting of a nucleobase sequence which shares at least 75% sequence identity with any of SEQ ID NOs 1-23 are also included as part of the invention. Alternatively, the amount of sequence identity the probe shows with the nucleobase sequence identified by SEQ ID NO can be approximately 80%, 85%, 90% or 95%. In other embodiments, the invention includes probes that consist of a contiguous segment of any of SEQ ID NOs 1-23, wherein those segments are at least 8 or at least 20 nucleobases long. In a further variation, the invention includes probes that consist of a contiguous segment, at least 8 or at least 20 nucleobases long, of any sequence at least 75% identical to any of SEQ ID NOs 1-23.

Probes comprising DNA mimics are part of the invention and are suitable for the assays described herein. DNA mimics include, for example, phosphorthioate oligonucleotides, peptide nucleic acids (PNAs), and locked nucleic acids (LNAs). Like nucleic acids, DNA mimics are spoken of as having a nucleobase sequence according to the A, C, G, T and U base portion of each of their respective monomer units. Probes comprising nucleic acid or probes comprising DNA mimics can be used in an assay, or combinations of any of the above probes can be used. DNA mimic portions and nucleic acid portions can be combined in one chimeric molecule (e.g., a PNA/DNA chimera), which is a variety of DNA mimic. For exemplary methods and compositions regarding PNA/DNA chimeras, see WO 96/408709.

Locked nucleic acid (LNA) and peptide nucleic acid (PNA) can be used in high affinity probes which can provide higher sensitivity and specificity than conventional DNA probes. Although LNA and PNA can employ common nucleobases (A, C, G, T, and U) and can hybridize to nucleic acids with sequence specificity according to Watson-Crick base pairing rules, they differ both structurally and functionally from DNA. Peptide nucleic acid, despite its name, is neither a peptide nor a nucleic acid, nor is it even an acid, but a non-naturally occurring polyamide backbone composed of (aminoethyl)-glycine subunits where the nucleobases are connected to the backbone by an additional methylene carbonyl moiety. See U.S. Pat. No. 5,539,082 and Egholm et al., Nature 365:566-568, 1993. As used herein, the term “peptide nucleic acid” or “PNA” means an oligomer, linked polymer or chimeric oligomer, comprising two or more PNA subunits (residues), including any of the polymers referred to or claimed as peptide nucleic acids in U.S. Pat. Nos. 5,539,082, 5,527,675, 5,623,049, 5,714,331, 5,736,336, 5,773,571, 5,786,461, 5,837,459, 5,891,625, 5,972,610, 5,986,053, 6,107,470 and 6,357,163. Due in part to its net neutral electrical charge, PNA can form hybrids rapidly and stably with naturally occurring nucleic acids. LNA is a DNA mimic created by chemically joining the 2′ oxygen and 4′ carbon of a ribonucleoside through a methylene linkage. The highly rigid structure of the resultant locked 3′-endo conformation reduces the conformational flexibility of the ribose. The increased rigidity and local organization of the LNA phosphate backbone lowers the entropic penalty for hybridization of LNA probes as compared to DNAs of the same relative composition. These structural features provide PNA and LNA probes with higher affinity for target sequences and furthermore allow PNA and LNA probes to hybridize under conditions that are destabilizing to naturally occurring nucleic acids, such as low salt concentration or the presence of guanidinium hydrochloride. These attributes enable PNA probes to access targets, such as highly structured rRNA and double stranded DNA, known to be inaccessible to DNA probes (Fuchs, Appl Envir Micro 64(12):4973-82, 1998). Therefore, probes comprising PNA or LNA analogs can be shorter than probes comprising only nucleic acids. The DNA mimic portion of a probe can be, for example, at least 8 nucleobases long, and can also be 30 or fewer nucleobases in length in some embodiments.

To determine the percent identity of two nucleobase sequences, the sequences are aligned for optimal comparison. In the simplest concept of identity, two nucleobase sequences are compared after aligning them for the maximum number of matches at the same position, without the introduction of any gaps. In a somewhat more complex concept of identity, the sequences are aligned and gaps can be introduced in one or both of a first and a second nucleobase sequence for optimal alignment, and non-homologous (dissimilar) sequences can be disregarded for comparison purposes. In a preferred embodiment, the length of a reference sequence aligned for comparison purposes is at least 40%, preferably at least 50%, more preferably at least 60%, and even more preferably at least 70%, 80%, or 90% of the length of the reference sequence. The nucleobases at corresponding nucleobase positions are then compared. When a position in the first sequence is occupied by the same nucleobase as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent similarity between two sequences can be accomplished using a mathematical algorithm. (Computational Molecular Biology, Lesk, A. M.,ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereaux, J., eds., M. Stockton Press, New York, 1991).

The nucleic acids of the present invention can be used as a “query sequence” to perform a search against databases to, for example, identify other family members or related sequences. Such searches can be performed using the software packages NCBI BLAST2, WU-BLAST 2.0, based on Altschul, et al. (J. Mol. Biol. 215:403-10 (1990)), or using FASTA, which offer default parameters. These programs can be used to align two known nucleobase sequences and to establish percentages of sequence identity and/or sequence similarity in a comparison of the two sequences.

The terms “complementary” or “complementarity” refer to the natural binding of the base portions of nucleic acids or DNA mimics under permissive salt and temperature conditions by base-pairing. Complementarity between two single-stranded molecules may be “partial” in which only some of the nucleic acids bind, or it may be complete when total complementarity exists between the single-stranded molecules (that is, when A-T and G-C base pairing is 100% complete). The degree of complementarity between nucleic acid strands has significant effects on the efficiency and strength of hybridization between nucleic acid strands.

The hybridization characteristics of a probe are usually described by the melting point (Tₘ) of the probe-target hybrid. The melting point is therefore an important parameter used to guide the experimentation described above to determine the suitable hybridization conditions. However, when an assay is dependent on simultaneous hybridization of two probes each of these two probes must be designed with similar hybridization characteristics such that the same hybridization conditions are suitable for both probes. The length of the nucleobase sequence provides a rough assessment of the hybridization characteristics, but can be refined by calculating the Tₘ. The degree of similarity between the hybridization characteristics of two probes is dependent on both the stringency of the hybridization conditions and the desired degree of discrimination that needs to be achieved.

Examples of hybridization conditions can be found on pages 2.10.1-2.10.16 (containing Supplements up through Supplement 42) and pages 6.3.1-6 (containing Supplements up through Supplement 24) in Current Protocols in Molecular Biology (Ausubel, F. M. et al., eds., containing Supplements up through Supplement 73, January, 2006). Examples of high, medium, and low stringency conditions can be found in the description on page 36, line 1 to page 37, line 12 of WO 98/40404.

Examples of stringency conditions are shown in Table 2 below. Highly stringent conditions are those that are at least as stringent as, for example, conditions A and B. Medium stringency conditions are at least as stringent as, for example, conditions C and D. Low stringency conditions are at least as stringent as, for example, conditions E and F.

TABLE 2

| | | Hybrid | | Wash |
|---|---|---|---|---|
| Stringency | Polynucleotide | Length | Hybridization Temperature | Temperature |
| Condition | Hybrid | (bp)^{‡} | and Buffer^{†} | and Buffer^{†} |
| A | DNA:DNA | ≧50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:DNA | ≧50 | 65° C.; 4xSSC -or- | 65° C.; 1xSSC |
| | | | 42° C.; 4xSSC, 50% formamide | |
| D | DNA:DNA | <50 | T_{H}*; 4xSSC | T_{H}*; 4xSSC |
| E | DNA:DNA | ≧50 | 50° C.; 4xSSC -or- | 50° C.; 2xSSC |
| | | | 40° C.; 6xSSC, 50% formamide | |
| F | DNA:DNA | <50 | T_{N}*; 6xSSC | T_{N}*; 6xSSC |

^{‡}The hybrid length is that anticipated for the hybridized region(s) of the hybridizing molecules. When hybridizing a probe to a target DNA of unknown sequence, the hybrid length is assumed to be that of the hybridizing probe. When molecules of known sequence are hybridized, the hybrid length can be determined by aligning the sequences of the molecules and identifying the region or regions of optimal sequence complementarity.
^{†}SSPE (1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete.
*T_{B}-T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1xSSC = 0.165 M).

Those of ordinary skill in the art of nucleic acid hybridization will recognize that factors commonly used to impose and/or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target sequence combination is often found by the well known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

Probes are a part of the invention, and can be used in assays to detect genes of interest. Probes comprise nucleic acids and/or DNA mimics as described above. Probes can further comprise a linker moiety, such as those that can chemically (which can be by covalent bonds or non-covalent interactions) link a nucleic acid molecule to a support. Other non-pairing moieties can be included in probes. A “capture probe” comprises any of the nucleic acids or DNA mimics described above and can be attached to a support, for example, by a linker moiety. A capture probe optionally also comprises a spacer moiety. The use of spacer moieties has been studied for the influence of steric hindrance and charge on the efficiency of hybridization reactions using immobilized oligonucleotides (Shchepinov, M. S. et al., Nucleic Acids Res. 25:1155-1161, 1997).

The function of a linker moiety and a spacer moiety can be combined. Examples of spacer/linker moieties for probes comprising DNA mimics are aminoalkyl carboxylic acids (e.g., aminocaproic acid) side chains of an amino acid (e.g. the side chain of lysine or ornithine) natural amino acids (e.g., glycine), aminooxyalkylacids (e.g., 8-amino-3,6-dioxaoctanoic acid), alkyl diacids (e.g., succinic acid) or alkyloxy diacids (e.g., diglycolic acid). Spacer/linker moieties may also be constructed to improve the water solubility of the probe.

Probes can comprise at least a nucleic acid or DNA mimic portion, and in addition, can comprise a label which can be used, for example, to facilitate detection of the probe and other molecules that may be bound to it. A label is a chemical moiety which is detectable under some condition. Labels can take a variety of forms, and are not limited by structure. Examples of detectable moieties suitable for use as labels include dextran conjugates, a branched nucleic acid detection system, chromophores, fluorochromes, spin labels, radioisotopes, mass labels, enzymes, haptens, chemiluminescent compounds, and substrates for enzymes that convert their substrates to a colored or fluorescent product.

A probe comprising a label is given the term “detector probe.” Such probes can be used in a sandwich type hybridization assay to hybridize to a region of a target nucleic acid to which the capture probe does not hybridize.

Labeling of a PNA is analogous to peptide labeling. Because the synthetic chemistry of assembly is essentially the same, any method commonly used to label a peptide can usually be adapted for use in labeling a PNA. Thus, PNAs may be labeled with numerous detectable moieties. Generally, a label which can be linked to a nucleic acid or peptide can be linked to a PNA.

Typically, the N-terminus of the PNA is labeled by reaction with a moiety having a carboxylic acid group or activated carboxylic acid group. One or more spacer moieties can be introduced between the labeled moiety and the PNA oligomer. Generally, the spacer moiety is incorporated prior to performing the labeling reaction. However, the spacer may be embedded within the label and thereby be incorporated during the labeling reaction. Specialized reagents can be attached to the PNA. For example, a terminal arylamine moiety can be generated by condensing a suitably protected 4-aminobenzoic acid derivative with the amino terminus of the PNA oligomer.

Labeling reagents can be supplied, for example, as carboxylic acids or as the N-hydroxysuccinidyl esters of carboxylic acids. Numerous amine reactive labeling reagents are commercially available (as, for example, from Molecular Probes, Eugene, Oreg.). Some suitable fluorochromes (fluorophores) include 5(6)-carboxyfluorescein (Flu), 6-((7-amino-4-methylcoumarin-3-acetyl)amino)hexanoic acid (Cou), 5(and 6)-carboxy-X-rhodamine (Rox), Cyanine 3 (Cy3) Dye, Cyanine 3.5 (Cy3.5) Dye, Cyanine 5 (Cy5) Dye, Cyanine 5.5 (Cy5.5) Dye, Cyanine 7 (Cy7) Dye, and Cyanine 9 (Cy9) Dye. Cyanine dyes 3, 3.5, 5 and 5.5 are available as NHS esters from Amersham (Arlington Heights, Ill.), or the Alexa dye series (Molecular Probes). Some suitable haptens include 5(6)-carboxyfluorescein, 2,4-dinitrophenyl, digoxigenin, and biotin. Enzymes to be used as labels include soybean peroxidase, alkaline phosphatase and horseradish peroxidase. Other suitable labeling reagents and preferred methods of attachment would be recognized by those of ordinary skill in the art of PNA synthesis.

The nucleobase sequences of the nucleic acids or DNA mimic portions of the probes and probe sets are selected to provide specificity for the assay while retaining some flexibility of the nucleobase sequence in the targeted region. Probes can hybridize to targets with some mismatch without significant loss of signal. Mismatch tolerance is attained by the use of probes with melting temperatures significantly above the imposed stringency of the assay conditions, and by careful positioning of probes to regions of known heterogeneity among suspected targets. The probes of this invention can bind to targets which are only 75% homologous in terms of Watson-Crick anti-parallel sequence alignments. Mismatch hybridization is generally considered unfavorable in assays requiring hybridization. Use of mismatch tolerant probes allows an assay to detect targets which may not have been sequenced or otherwise characterized. The use of probe sets imparts specificity to the assay by requiring that target regions of at least one capture probe, and at least one detector probe be present coincidentally on a single nucleic acid molecule in the sample. Furthermore, probe sets of this invention are directed to regions which are in close proximity to each other in the sample DNA, generally within 1000 base pairs. Probe sets may be used wherein probes target abutting regions of the target, with no bases between the target regions to as many as 1000 bases between target regions.

Some assays of the invention may use sample processing techniques which shear, or otherwise reduce sample nucleic acid targets to small pieces. Target reducing may be achieved enzymaticly, for example, with restriction enzymes, chemically, for instance with high pH, physically, for instance through the use of high temperatures, or mechanically, for example, through sonication or the application of shearing forces. A complex genome of many millions of nucleotides may be “decomplexed” by reducing the average length of the molecules in the sample to thousands or hundreds of nucleotides. Probe sets which are directed to proximal regions of reduced targets have a greater likelihood of binding (and detecting) the target. Though inherent to any probe based assay, it is worth noting that probe design must always include safeguards to avoid the possibility of probe-probe interactions, and probe self-compatibility. Also, probe sets frequently require careful titration to achieve maximum signal to noise ratios.

Though the probes are tolerant of mismatches, high target specificity can be achieved through a) the use of sets of probes with sequences chosen from sites that are in close proximity to each other on the target nucleic acid; b) reduction of the size of the nucleic acid molecules in the sample; c) careful titration of probe concentration; and d) design of probes directed to target regions which are sufficiently unique.

Supports suitable for the attachment of probes include, for example, organic polymers such as polystyrene, polyethylene, polypropylene, polyfluoroethylene, polyethyleneoxy, and polyacrylamide, as well as co-polymers and grafts thereof. A support may be made of nylon, derivativized nylon (see, for example, Van Ness, J. et al., Nucleic Acids Res. 19:2245-3350, 1991), or DNA-BIND™ surface (Corning, Corning, N.Y.) in which aminated oligonucleotides bind in a reaction with N-oxysuccinimide. A support may also be inorganic, such as glass, silica, controlled-pore-glass (CPG), or reverse-phase silica. Epoxide-coated glass slides, amino-silane coated glass slides, nitrocellulose, Immobilon®, and polytetrafluoroethylene are also examples of supports.

The configuration of a support may be in the form of beads, spheres, particles, granules, a gel, or a surface. Surfaces may be planar, substantially planar, or non-planar. Solid supports may be porous or non-porous, and may have swelling or non-swelling characteristics. A support may be configured in the form of a well, depression or other container, vessel, feature or location mapped on a surface. A plurality of supports may be configured in an array at various locations, addressable for robotic delivery of reagents, or by detection means including scanning by laser illumination and confocal or deflective light gathering.

As used herein, “attached to a support” means immobilized on or to a support, which in some embodiments, are solid supports. It is understood that immobilization can occur by any means, including for example; by covalent bonds, by ionic bonds, by hydrogen bonds, by hydrophobic interactions, by electrostatic immobilization, by attachment through a ligand/ligand interaction, by contact or by depositing on the surface, as in “printing” spots of liquid on a glass slide or other planar surface to form an array or microarray.

Arrays are surfaces to which two or more probes have been immobilized, each at a specified position on a support. Usually, multiple probes are attached to a support in a defined pattern. The nucleobase sequences of the nucleic acid or DNA mimic portions of the immobilized probes can be judiciously chosen to interrogate a sample of genomic DNA that may contain several target genes of interest. Because the location and composition of each immobilized probe is known, arrays can be useful for the simultaneous detection, identification and/or quantitation of multiple target genes of interest that are present in the sample.

The probes of the invention are useful in carrying out methods to detect genes of interest (“target” genes) in samples containing bacteria, especially staphylococci, and especially Staphylococcus aureus. The methods rely on the ability of single stranded nucleic acids or DNA mimics to form hydrogen bonds with single-stranded nucleic acids with a complementary or nearly complementary nucleobase sequence through base pairing, thereby forming double stranded “hybrid” nucleic acids. Many assays and variations of these assays have been devised to test the complementarity of probes and the DNA to be tested, thereby allowing the detection of the presence or absence of the target gene, and, in some cases, allowing for the quantitation of the gene, if present. Available methods have made use of PCR (polymerase chain reaction) or other amplification methods to increase the amount of target gene nucleic acid available for hybridization. Other methods do not incorporate an amplification step.

A method for the detection of genes in a sample of DNA has been described (Skov, R. L. et al., Journal of Antimicrobial Chemotherapy 43:467-475, 1999; Levi, K. and K. J. Towner, J. Clin. Microbiol. 41:3890-3892, 2003). This method is a “sandwich” design hybridization assay in which DNA hybridizes to both capture probe and detector probe, and does not require amplification of the DNA to be tested. A Staphylococcus strain is grown in culture, the cells are concentrated, if grown in liquid culture, and lysed. The lysate is vortexed, and heated to denature the DNA. Detector probe(s) and aliquots of the DNA sample are distributed among wells coated with capture probe(s), and the capture probe(s), DNA fragments, and detector probe(s) are incubated with agitation, thereby allowing hybridization. The wells are washed, and a detection method is applied so that hybridized detector probe can be detected. Kits to perform this method are available (EVIGENE™; AdvanDx, Woburn, Mass.).

A method previously described is multiplex PCR (Monecke and Ehricht, Clin. Microbiol. Infect. 11:825-833, 2005; Pichon, J-P, B. Bonnarud, P. Cleuziat and F. Mallet, Nucleic Acids Research 34(6):e46 (10 pages). Real-time PCR (e.g., Fang and Hedin, J. Clin. Microbiol. 41:2894-2899, 2003) relies on the detection and quantitation of a fluorescent reporter during amplification. Several types of fluorescence monitoring systems have been described, including hydrolysis probes containing fluorescent dye as well as a quenching dye (TaqMan® probes), hybridizing probes designed to fluoresce during the annealing step (molecular beacon probes; see, e.g., Sinsimer, D. et al., J. Clin. Microbiol. 43:4585-4591, 2005), and DNA binding agents such as the SYBR-green DNA binding dye. Further methods for detection of genes have been described.

Kits for the characterization of Staphylococcus are other embodiments of the invention. The kits can comprise one or more oligonucleotides or DNA mimics that can be used as primers to amplify bacterial DNA purified or partially purified from, for example, a patient specimen, a bacterial culture or a colony of bacteria which may be suspected of containing staphylococci. In a different embodiment, the kits can comprise one or more capture probes, one or more detector probes, or one or more sets comprising one or more capture probe(s) and one or more detector probe(s) to be used together. In the case of a kit comprising one or more capture probes, the capture probes can be supplied attached to a support, for example, wells of a plate designed to hold small volumes of liquid. The kits can comprise, for instance, as other components, lysis solution, wash solution, and reagent(s) for producing a detectable product from the label of a detector probe.

The assays described herein can be used to characterize the genotype of bacteria, wherein the genotype includes all genetic material located in the cells, which encompasses the bacterial chromosome as well as any extrachromosomal elements. The bacteria can be tested directly, or can be propagated from a sample of tissue, or other collected material such as sputum, laryngeal swabs, gastric lavage, bronchial washings, biopsies, aspirates, expectorates. The bacteria can also be tested directly, or propagated from body fluids (for example, cerebrospinal fluid, pericardial fluid, synovial fluid, blood, pus, amniotic fluid, urine, wound washings, and mucous). The sample can be taken from a person or animal known or suspected of having a staphylococcal infection. The sample can be taken from a person or animal suspected of being a carrier of Staphylococcus aureus or other species of Staphylococcus. In some cases, the bacteria to be used as the source of DNA have been isolated to colonies and have been characterized as Staphylococcus aureus or another species of Staphylococcus. However, this isolation and characterization is not necessary to carry out the assays.

A Staphylococcus strain is a group of cells comprising cells of the genus Staphylococcus. In one case, the cells have undergone some process of genetic purification, such as isolation of a colony. In another case, the cells can be uncharacterized as to species, and are not necessarily pure in terms of species.

Genes conferring traits for antibiotic resistance or virulence markers may be present in a cell as part of the extrachromosomal genome. Non-exclusive examples of components of the extrachromosomal genome include plasmids and viruses. Genes conferring vancomycin resistance and mupirocin resistance, for instance, can be found in plasmids. Extrachromosomal genetic elements can be acquired by a cell through lateral mechanisms including conjugation or infection by, for instance, a bacteriophage. Movement of chromosomal cassettes associated with methicillin resistance between Staphylococcus cells, and between genera is suspected to be phage mediated (Hanssen et al., FEMS Immunol Med Microbiol. 2006 Feb., 46(1):8-20). Extrachromosomal genetic elements may be present in a cell for its entire life cycle, or they may be acquired or lost during the life cycle of the cell. In some cases, cells may contain variants of a particular gene which are not colocated. The gene encoding exfoliative toxin A (eta) of Staphylococcus aureus, for instance, is located chromosomally, whereas, the gene encoding the B variant (etb) is found in, and transferred via a plasmid. It has been suggested that etb is more strongly associated with the condition known as staphylococcal scaled-skin syndrome (SSSS) (see Yamasaki et al. JCM April 2005).

Table 3 shows genes of Staphylococcus aureus and their GenBank Accession numbers.

TABLE 3
GenBank Accession Numbers

| | | Accession |
|---|---|---|
| Gene | Gene Name | Number |
| nuc | Staphylococcal nuclease | V01281 |
| mecA | penicillin binding protein 2′ | X52592 |
| vanA | vancomycin/teicoplanin A-type | AE017171 |
| | resistance protein | |
| vanB | D-alanine ligase | L06138 |
| tst | toxic shock syndrome toxin-1 | AY074881 |
| lukS-PV | Panton-Valentine leukocidin | X72700 |
| lukF-PV | Panton-Valentine leukocidin | X72700 |
| ileS-2 | isoleucyl tRNA-synthetase (mupA) | X75439 |

### EXAMPLES

### Example 1

### Probes

Probes are displayed in the 5′ to 3′ orientation. Detector probes are labeled with five ligand molecules at the 3′ end. Capture probe P-Cpl: AAA-TCT-GAG-AAA-TTT-TTA-ACT-TAT-CGG-AAT-CTG-ATG-TTG-C (SEQ ID NO:9). Capture probe P-CP2: CCA-GCA-GCT-TTG-AGT-ATT-AAT-GTA-TCT-TTA-TCA-TAA-CTT-T (SEQ ID NO:10). Detector Probe P-Dt2: AAA-TTG-AGA-ACT-AAT-AGT-GTC-TTT-TGG-ATT-TGG-CTT-TGT-A (SEQ ID NO:11).

### Bacterial Strains

Methicillin-susceptible S. aureus (MSSA) ATCC 6538 (#1, mecA & PVL negative) and methicillin-resistant S. aureus (MRSA) ATCC 33591 (#2, mecA positive & PVL negative) were used as negative control strains. An MRSA SCCmec type IV (#15) was used as a positive control strain according to earlier genotypic testing provided by Statens Serum Institut, Copenhagen, Denmark.

### Hybridization Assay

Samples were prepared and assayed as described by Skov et al., 1999. In brief, 2 μL bacteria were suspended in 100 μL of a lysis solution (Reagent A) and incubated for 20 min at 37° C. After incubation 50 μL of a second lysis solution (Reagent B) was added and incubated two times at 100° C. for 15 min with brief vortexing between incubations. The detector probe P-Dt2 (5 nM) was added to the wells coated with either capture probe P-Cp1, or P-Cp2 (60 nM). The wells were incubated at 50° C. at 400 rpm in an ELISA incubator/shaker. The wells were washed one time with 200 μL washing solution before anti-ligand enzyme conjugate was added to each well. After incubation with conjugate for 30 min at 37° C., the wells were washed four times with wash solution (200 μL) and pNPP-substrate was added to each well followed by incubation for 40 min at 37° C. Stop solution was added to each well after which they were inspected for a color change indicative of enzyme activity by reading on a spectrophotometer at 405 nm.

### Results

The results of Example 1 are presented in Table 4, which displays the absorbance values obtained at 405 nm for samples tested with either capture probe P-Cp1 (column II), or P-Cp2 (column III). In row B, both probe combinations tested show only a background level of signal in the absence of cells in the lysis step. Likewise, in rows C and D where strains were tested which are known to not contain the PVL genes, only very weak signals were seen, just above background. In row E, use of a strain known to be PVL positive shows a dramatic increase in signal with either capture probe tested.

TABLE 4

| | I | II | III |
|---|---|---|---|
| A | SAMPLE | P-Cp1 | P-Cp2 |
| B | Buffer Only | 0.231 | 0.220 |
| C | MSSA (ATCC 6538) | 0.274 | 0.302 |
| D | MRSA (ATCC 33591) | 0.256 | 0.321 |
| E | PVL-positive MRSA (#15) | 3.105 | 3.084 |

### Example 2

### Probes

Probes were the same as in Example 1. However, only capture P-Cp2 (60 nM) was used and detector probe P-Dt2 was added to the wells at a concentration of 1 nM.

### Bacterial Strains

Methicillin-susceptible S. aureus (MSSA) ATCC 6538 (#1, mecA & PVL negative) and methicillin-resistant S. aureus (MRSA) ATCC 33591 (#2, mecA positive & PVL negative) were used as negative control strains. Seven strains typed by Statens Serum Institut (Copenhagen, Denmark) were used, including six PVL negative MRSA of various SCCmec types (strains #9-14) and one PVL positive SCCmec (#15). Three coagulase-negative staphylococci (CNS, #3, #4 & #8), one Enterococcus faecalis (#5, vanB positive), one Enterococcus faecium (#6, vanA positive) and one Enterococcus casseliflavus (#7) were also investigated.

### Hybridization Assay

Sample preparations were done as in Example 1, except the lysis solutions and the other solutions were added drop-wise from dropper bottles to achieve approximately the same volumes. The samples and detector probes were incubated in capture probe coated plates for 1 hour at 55° C. at 400 rpm in an ELISA incubator/shaker. After incubation with conjugate for 30 min at 37° C., the wells were washed four times with wash solution and substrate was added to each well followed by incubation for 15 min at room temperature. Stop solution was added to each well after which they were inspected for a color change indicative of enzyme activity by reading on a spectrophotometer at 490 nm.

A second test was performed to assess the presence of the mecA gene in the samples using the same assay format as the PVL assay, though with different capture and detector probes. The mecA gene encodes the beta-lactam-inducible penicillin-binding protein (PBP-2′; also known as PBP-2a) which confers methicillin resistance in S. aureus and other staphylococci. The vanA and vanB genes encode vancomycin resistance proteins.

### Results

The results of Example 2 are presented in Table 5, which displays the sample type in column I, the sample number in column II, the raw absorbance values obtained at 490 nm for samples tested in column III, and the absorbance of samples minus buffer control in column IV. To obtain the values in column IV the buffer control value (row B, column III, 0.097) is subtracted from the raw RLU values in column III. RLU-Bkg values are used to help to discriminate real signal from sample-to-sample baseline variation. Column V of Table 5 displays the values obtained from an assay performed to assess the presence of the mecA gene in these samples.

With reference to Table 5 column IV, there is only one sample, sample 15, which demonstrates signal significantly above the baseline. All other samples have signals which are not significantly above the noise. Sample 15 contains the PVL-positive S. aureus strain; none of the other samples are PVL positive. This example demonstrates that the assay as described in Example 1 was adapted to a different protocol without negatively affecting the assay results. Also, since the protocol was converted from exact volume additions in Example 1 (performed with a pipette) to less exact dropper additions, the assay can be described as being robust. This example also demonstrates that strains positive for other virulence genes such as mecA, vanA and vanB will not produce a positive result by the PVL assay.

With reference to column V, the data are presented for the mecA test. The test was performed using exactly the same assay format as the PVL assay, though with different capture and detector probes. Column V, rows D, F, K, L, M, N, O, P, and Q all demonstrate strong signals whereas the other rows demonstrate signals less than or equal to the buffer control (row B). The same rows that have a strong signal in column V were also all previously genotyped as mecA positive. The mecA and PVL tests were performed on the same samples and in same assay format but otherwise are independent of each other. These data demonstrate that assessments of both the PVL and the mecA gene can be performed in an array format.

TABLE 5

| | | II | | | |
|---|---|---|---|---|---|
| | I | Sample/ | III | IV | V |
| A | Sample Type | Strain No. | Raw RLU | RLU-Bkg | mecA |
| B | Buffer Control | | 0.097 | 0.000 | 0.330 |
| C | PVL-negative MSSA | 1 | 0.108 | 0.011 | 0.130 |
| D | PVL- negative MRSA | 2 | 0.209 | 0.112 | 1.467 |
| E | CNS | 3 | 0.101 | 0.004 | 0.148 |
| F | CNS (mecA positive) | 4 | 0.107 | 0.010 | 1.332 |
| G | E. faecalis (vanB positive) | 5 | 0.100 | 0.003 | 0.119 |
| H | E. faecium (vanA positive) | 6 | 0.113 | 0.017 | 0.104 |
| I | E. casseliflavus | 7 | 0.096 | 0.000 | 0.111 |
| J | CNS | 8 | 0.110 | 0.013 | 0.143 |
| K | PVL-negative SCCmec type I | 9 | 0.114 | 0.018 | 2.496 |
| L | PVL-negative SCCmec type Ia | 10 | 0.101 | 0.005 | 2.282 |
| M | PVL-negative SCCmec type II | 11 | 0.100 | 0.003 | 1.301 |
| N | PVL-negative SCCmec type III | 12 | 0.096 | −0.001 | 0.955 |
| O | PVL-negative SCCmec type IIIa | 13 | 0.101 | 0.005 | 1.014 |
| P | PVL-negative SCCmec type IV | 14 | 0.110 | 0.014 | 1.224 |
| Q | PVL-positive SCCmec type IV | 15 | 1.577 | 1.480 | 1.115 |

### Example 3

### Probes

Probes are displayed in the 5′ to 3′ orientation. Detector probes are labeled with five ligand molecules at the 3′ end. Capture probe tsst -Cp1 (60 nM): GCT-ACA-GAT-TTT-ACC-CCT-GTT-CCC-TTA-TCA-TCT-AAT-CA (SEQ ID NO:6). Capture probe tsst -Cp2 (60 nM): GCA-TCT-ACA-AAC-GAT-AAT-ATA-AAG-GAT-TTG-CTA-GAC-TGG-T (SEQ ID NO:7). Detector Probe tsst -Dt1 (5 nM): GCA-TCT-ACA-AAC-GAT-AAT-ATA-AAG-GAT-TTG-CTA-GAC-TGG-T (SEQ ID NO:7). Detector Probe tsst -Dt2 (5 nM): TAG-TGA-AGT-TTT-AGA-TAA-TTC-CTT-AGG-ATC-TAT-GCG-AT-A (SEQ ID NO:8).

### Bacterial Strains

Methicillin-susceptible S. aureus (MSSA) ATCC 6538 (#1, mecA & tst negative) and methicillin-resistant S. aureus (MRSA) ATCC 33591 (#2, mecA positive & tst negative) were used as negative control strains. An MSSA (#70) was used as a tst positive control strain according to earlier genotypic testing provided by Statens Serum Institut, Copenhagen, Denmark. Three coagulase-negative staphylococci (CNS, #3, #4 & #8), one Enterococcus faecalis (#5, vanB positive), one Enterococcus faecium (#6, vanA positive) and one Enterococcus casseliflavus (#7) were also investigated.

### Hybridization Assay

Sample preparations were done as in Example 2 except the wells were washed three times with 200 μL washing solution before anti-ligand enzyme conjugate was added to each well. After incubation with conjugate for 30 min at 37° C., the wells were washed four times with wash solution and substrate was added to each well followed by incubation for 15 min at room temperature. Stop solution was added to each well after which they were inspected for a color change indicative of enzyme activity by reading on a spectrophotometer at 490 nm.

### Results

The results of Example 3 are presented in Table 6, which displays the sample type in column I, the sample number in column II the raw absorbance values obtained at 492 nm for samples tested with either capture probe tsst-Cp1 and detector probe tsst-Dt1 (column III), or tsst-Cp2 and tsst-Dt2 (column IV).

With reference to Table 6, row K, there is only one sample, sample 70, which demonstrates signal significantly above the baseline with either capture and detector probes tested. All other samples have signals which are not significantly above the noise (buffer control). Sample 70 contains the tst-positive S. aureus strain; none of the other samples are tst positive. This example demonstrates that strains positive for other virulence genes such as mecA, vanA and vanB will not produce a positive result by the TSST-1 assay.

TABLE 6

| | I | II | III | IV |
|---|---|---|---|---|
| | Sample | Sample/ | tsst-Cp1 + | tsst-Cp2 + |
| A | Type | Strain No. | tsst-Dt1 | tsst-Dt2 |
| B | Buffer Control | | 0.214 | 0.195 |
| C | Tst-negative MSSA | 1 | 0.222 | 0.127 |
| D | Tst-negative MRSA | 2 | 0.173 | 0.121 |
| E | CNS | 3 | 0.142 | 0.190 |
| F | CNS (mecA positive) | 4 | 0.136 | 0.186 |
| G | E. faecalis (vanB positive) | 5 | 0.138 | 0.201 |
| H | E. faecium (vanA positive) | 6 | 0.154 | 0.165 |
| I | E. casseliflavus | 7 | 0.223 | 0.244 |
| J | CNS | 8 | 0.181 | 0.253 |
| K | Tst-positive MSSA | 70 | 2.182 | 2.102 |

### Example 4

### Probes

Probes were the same as in Examples 2 & 3. However, only capture tsst-Cp2 (60 nM) was used and detector probe tsst-Dt2 was added to the wells at a concentration of 5 nM.

### Bacterial Strains

Methicillin-susceptible S. aureus (MSSA) ATCC 6538 (#1, meca, PVL, tst negative), methicillin-resistant S. aureus (MRSA) ATCC 33591 (#2, mecA positive & PVL, tst negative) and two coagulase-negative staphylococci (CNS, #3, mecA, PVL, tst negative & CNS, #4, mecA positive & PVL, tst negative) were used as negative control strains. Two strains typed by Statens Serum Institut (Copenhagen, Denmark) were used, including one PVL positive SCCmec (#15) and one tst positive MSSA (#70). One MRSA (#75) tst positive strain typed by O. Denis, Université Libre de Bruxelles, Brussels, Belgium, was also investigated.

### Hybridization Assay

Sample preparations were done as in Example 3. The samples and detector probes were incubated in capture probe coated plates for 1 hour at 55° C. at 400 rpm in an ELISA incubator/shaker. After incubation with conjugate for 30 min at 37° C., the wells were washed four times with wash solution and substrate was added to each well followed by incubation for 15 min at room temperature. Stop solution was added to each well after which they were inspected for a color change indicative of enzyme activity by reading on a spectrophotometer at 492 nm.

The samples were tested for the presence of the mecA, nuc, tst and PVL genes using the same assay format as the PVL and tst assay, though with different capture and detector probes. The nuc gene encodes staphylococcal nuclease, and is used as a positive indicator of S. aureus.

### Results

The results of Example 4 are presented in Table 7. With reference to Table 7, sample type in column I, the sample number in column II, the data are presented for the PVL in column III, the data are presented for the tst test in column IV, the data are presented for the nuc test in column V and the data are presented for the mecA test in column VI. All data from column III to VI are raw absorbance values obtained at 492 nm.

The PVL-positive SCCmec type IV sample (row G) showed a strong signal for PVL, whereas the other samples showed signals less than or equal to the buffer control (row B). The sample type that gave a strong signal in column III was also previously genotyped as nuc and mecA positive. Column IV (tst), row H & I demonstrated strong signal whereas the other rows demonstrated signals less than or equal to the buffer control (row B). The mecA, nuc, tst and PVL tests were performed on the same samples and in the same assay format but otherwise were independent of each other. These data demonstrate that assessments of the PVL, tst, nuc and the mecA genes can be performed in an array format.

TABLE 7

| | | II | | | | |
|---|---|---|---|---|---|---|
| | I | Sample/ | III | IV | V | VI |
| A | Sample Type | Strain No. | PVL | tst | nuc | mecA |
| B | Buffer Control | | 0.208 | 0.116 | 0.237 | 0.159 |
| C | PVL, tst-negative MSSA | 1 | 0.122 | 0.090 | 1.482 | 0.113 |
| D | PVL, tst- negative MRSA | 2 | 0.108 | 0.105 | 1.868 | 1.643 |
| E | CNS | 3 | 0.106 | 0.119 | 0.259 | 0.142 |
| F | CNS (mecA positive) | 4 | 0.141 | 0.125 | 0.135 | 1.577 |
| G | PVL-positive SCCmec type IV | 15 | 1.497 | 0.108 | 1.851 | 1.702 |
| H | PVL-negative, tst-positive MSSA | 70 | 0.329 | 1.975 | 1.548 | 0.118 |
| I | PVL-negative, tst-positive MRSA | 75 | 0.155 | 1.810 | 1.928 | 1.481 |

### Example 5

### Probes

Probes were the same as in Example 4; however, for detection of PVL genes, a single capture probe, P-Cp3 (60 nM), and a single detection probe, P-Dt4 (1 nM) were used. P-Cp3 corresponds to SEQ ID NO:4 and P-Dt4 corresponds to SEQ ID NO:5. These probes are the complements of P-Cp2, and P-Dt2 respectively which were used in Example 4. Use of these probes demonstrated that the assay can be designed to detect either strand of a double-stranded target, and that probe sets in different parts of the array do not need to be directed against the same strand. Also included in this assay were the vanA and vanB probe set from a commercially available assay.

### Bacterial Strains

Methicillin-susceptible S. aureus (MSSA) ATCC 6538 (#1, mecA, PVL, tst negative), methicillin-resistant S. aureus (MRSA) ATCC 33591 (#2, mecA positive & PVL, tst negative) and two coagulase-negative staphylococci (CNS, #3, meca, PVL, tst negative & CNS, #4, mecA positive & PVL, tst negative) were used as negative control strains. Two strains typed by Statens Serum Institut (Copenhagen, Denmark) were used, including one PVL positive SCCmec (#15) and one tst positive MSSA (#70). One Enterococcus faecalis (#5, vanB positive), one Enterococcus faecium (#6, vanA positive) and one Enterococcus casseliflavus (#7) were also investigated.

### Hybridization Assay

The samples were tested for the presence of the mecA, nuc, tst, vanA/B and PVL genes using the same assay format as above. Sample preparations were done as in Example 3. The samples and detection probes were incubated in capture probe coated plates for 1 hour at 55° C. at 400 rpm in an ELISA incubator/shaker. After incubation with conjugate for 30 min at 37° C., the wells were washed four times with wash solution and substrate was added to each well followed by incubation for 15 min at room temperature. Stop solution was added to each well after which they were inspected for a color change indicative of enzyme activity by reading on a spectrophotometer at 492 nm.

### Results

The results of Example 5 are presented in Table 8. With reference to Table 8, Sample Type in column I, the Sample Number in column II, the data are presented for the PVL test in column III, the data are presented for the tst test in column IV, the data are presented for the nuc test in column V, the data are presented for the mecA test in column VI, and the data for the vanA/B test are presented in Column VII. All data from column III to VII are raw absorbance values obtained at 492 nm. The tests were performed using exactly the same assay format as the PVL and tst assays, though with different capture and detector probes.

Column III (PVL), row F demonstrated strong signal indicating the presence of the PVL genes whereas the other rows demonstrated low, near baseline, signals. The row that has a strong signal in column III was also previously genotyped as nuc and mecA positive. As in the previous example, Column IV (tst), row G demonstrated strong signal indicative of tst positivity. The meca, nuc, tst, van A/B and PVL tests were performed on the same samples and in same assay format but otherwise are independent of each other. These data demonstrate that assessments of the PVL, tst, nuc, van A/B and the mecA gene can be performed in an array format, and a single capture probe and detector probe are useful for detection of PVL. Though no vanA/B positive staphylococci were tested in this assay, the positive results from the Enterococci tested (rows H-J) demonstrate that the test should work equally well on vanA/B positive staphylococci.

TABLE 8

| | | II | | | | | |
|---|---|---|---|---|---|---|---|
| | I | Sample/ | III | IV | V | VI | VII |
| A | Sample Type | Strain No. | PVL | tst | nuc | mecA | vanA/B |
| B | PVL, tst-negative MSSA | 1 | 0.221 | 0.140 | 2.216 | 0.137 | 0.120 |
| C | PVL, tst- negative MRSA | 2 | 0.097 | 0.143 | 2.215 | 2.001 | 0.147 |
| D | CNS | 3 | 0.094 | 0.142 | 0.150 | 0.174 | 0.147 |
| E | CNS (mecA positive) | 4 | 0.105 | 0.141 | 0.175 | 1.772 | 0.136 |
| F | PVL-positive SCCmec type IV | 15 | 1.071 | 0.156 | 1.413 | 1.104 | 0.215 |
| G | PVL-negative, tst-positive MSSA | 70 | 0.261 | 0.979 | 1.075 | 0.203 | 0.249 |
| H | E. faecalis (vanB positive) | 5 | 0.137 | 0.153 | 0.142 | 0.103 | 1.846 |
| I | E. faecium (vanA positive) | 6 | 0.140 | 0.148 | 0.142 | 0.105 | 1.802 |
| J | E. casseliflavus | 7 | 0.155 | 0.236 | 0.159 | 0.128 | 0.137 |

### Example 6

### Probes

Probes were the same as in Example 2; however, the capture probe, P-Cp2 was replaced with a version containing LNA bases. The LNA capture probe, LP-Cp2, is identical in sequence to P-Cp2, but is 8 bases shorter (truncated from the 3′end), and contains LNA residues at positions 6, 9, 12, 15, 18, 21, 24, 27, and 32 of the original sequence (SEQ ID NO:4) which is otherwise constructed of DNA bases. LP-Cp2 was used at 60 nM. As in Example 2, P-Dt2 was used at 1 nM.

### Bacterial Strains

Clinical isolates of methicillin-resistant S. aureus strains were tested including two which were previously identified as PVL positive, #30 and #34, and one PVL negative, #31.

### Hybridization Assay

Sample preparations were done as in Example 3. The samples and detection probes were incubated in capture probe coated plates for 1 hour at 55° C. at 400 rpm in an ELISA incubator/shaker. After incubation with conjugate for 30 min at 37° C., the wells were washed four times with wash solution and substrate was added to each well followed by incubation for 15 min at room temperature. Stop solution was added to each well after which they were inspected for a color change indicative of enzyme activity by reading on a spectrophotometer at 490 nm. The samples were tested for the presence of the PVL genes using the same assay format as above.

### Results

Data are presented in Table 9. With reference to Table 9, the table displays Sample Type in column I, and absorbance data in column II, samples are displayed in rows B-E. The data demonstrate that use of the LNA capture probe results in higher signals for samples positive for the PVL genes (rows D and E) as compared to PVL negative or buffer only controls (rows C and B respectively).

TABLE 9

| | I | II |
|---|---|---|
| A | SAMPLE | LP-Cp2 |
| B | Buffer Only | 0.343 |
| C | PVL-negative MRSA (#31) | 0.457 |
| D | PVL-positive MRSA (#30) | 0.686 |
| E | PVL-positive MRSA (#34) | 0.762 |

### Example 7

Detection of the ileS-2 Gene in Mupirocin Resistant Staphylococcus aureus

Staphylococcus aureus cells are prepared and processed as described in Example 3, with the exception that capture probe M-Cp1 and detector probe M-Dt1 are used. The nucleobase sequence of M-Cp1 is CTC-ATT-GTT-GGA-GAT-GTG-GTA-ATC-CTT-TGA-TAT-ATT-ATG (SEQ ID NO:17) and of M-Dt1 is CAA-TAA-TAA-TAA-TAT-AGA-GTG-GTT-TCC-TTC-TCA-TAT-TAA-G (SEQ ID NO:18) (presented in the 5′-3′ orientation). M-Cp1 is used at 60 nM; M-Dt1 is used at 5 nM. As described above, the detector probe is labeled with one or more detectable moieties.

Presence of the detector probe is used to determine presence of the ileS-2 gene. Signal levels significantly above signal from one or more negative controls are considered positive and mupirocin resistance is inferred.

### Example 8

### Probes

Some of the probes were the same as in Example 5; however, the capture probe, P-Cp3 was replaced with a shorter version P-Cp4: 5′- GA TAA AGA TAC ATT AAT ACT CAA AGC TGC TGG-3′ (SEQ ID NO:21) and the detection probe P-Dt4 with a shorter P-Dt5: 5′CAA AGC CAA ATC CAA AAG ACA CTA TTA GTT CTC 3′ (SEQ ID NO:22). In this example two detection probes P-Dt5 and P-Dt6 were used together. The nucleobase sequence of P-Dt6 is 5′CAG ATT CTA ATG ACT CAG TAA ACG TTG TAG 3′ (SEQ ID NO:23). The capture probe, P-Cp4, is identical in sequence to P-Cp3, but is 7 bases shorter (truncated from the 5′end). The detection probe, P-Dt5, is identical in sequence to P-Dt4, but is 8 bases shorter (truncated from the 5′ and 3′ end). P-Cp4 was used at 60 nM. As in Example 6, P-Dt5 and P-Dt6 were used at 5 nM each. Presence of a second detector probe is used to determine presence of PVL in low target concentrations. The use of shorter capture and detection probes demonstrated that the assay can be designed with shorter probes.

### Bacterial Strains

Clinical isolates of S. aureus strains were tested including 3 which were previously identified as PVL positive, #155, #322 and #365 and two PVL negative, #156 and #28.

### Hybridization Assay

Sample preparations were done as in Example 6. The samples and detection probes were incubated in capture probe coated plates for 1 hour at 55° C. at 400 rpm in an ELISA incubator/shaker. After incubation with conjugate for 30 min at 37° C., the wells were washed four times with wash solution and substrate was added to each well followed by incubation for 30 min at room temperature. Stop solution was added to each well, after which they were inspected for a color change indicative of enzyme activity by reading on a spectrophotometer at 492 nm. The samples were tested for the presence of the PVL genes using the same assay format as above.

### Results

Data are presented in Table 10. With reference to Table 10, the table displays Sample Type in column I, and absorbance data in columns III to VI; samples are displayed in rows B-I. The data demonstrate that use of two detection probes results in higher signals for samples positive for the PVL genes with target concentration (rows E-IV, F-IV, F-VI and F-VI) as compared to one detection probe (rows E-III, F-III, E-V and F-V respectively).

Column V and VI (P-Cp4, shorter capture probe) demonstrated the same signal as Column III and IV (P-Cp3) indicating the use of shorter capture probes.

TABLE 10

| | | | III | IV | V | VI |
|---|---|---|---|---|---|---|
| | | II | P-Cp3 | | P-Cp4 | |
| | I | Sample/Strain | 5 nM | 5 nM P-Dt5 + | 5 nM | 5 nM P-Dt5 + |
| A | Sample type | No. | P-Dt4 | 5 nM P-Dt6 | P-Dt4 | 5 nM P-Dt6 |
| B | Buffer | | 0.152 | 0.121 | 0.148 | 0.204 |
| C | PVL-negative | 156 | 0.104 | 0.133 | 0.105 | 0.101 |
| | MSSA | | | | | |
| D | PVL-positive | 155 | 2.518 | 2.719 | 2.471 | 2.954 |
| | MSSA | | | | | |
| E | PVL-positive | 1:50 of 155 | 0.519 | 1.274 | 0.441 | 1.860 |
| | MSSA | | | | | |
| F | PVL-positive | 1:10 of 155 | 2.083 | 2.284 | 1.895 | 2.675 |
| | MSSA | | | | | |
| G | PVL-negative | 28 | 0.114 | 0.116 | 0.099 | 0.101 |
| | MRSA | | | | | |
| H | PVL-positive | 322 | 2.622 | 2.893 | 2.374 | 3.056 |
| | MRSA | | | | | |
| I | PVL-positive | 365 | 2.222 | 2.945 | 2.450 | 3.110 |
| | MRSA | | | | | |

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for detecting the lukS-PV and/or lukF-PV genes of a Staphylococcus in a sample, the method comprising hybridizing DNA of the sample to one or more capture probe(s) and one or more detector probe(s), wherein the capture probe(s) hybridize to a segment of the lukS-PV and/or lukF-PV genes, and the detector probe(s) hybridize to a segment of the lukS-PV and/or lukF-PV genes, and detecting or not detecting hybridized detector probe(s), thereby determining the presence or absence, respectively, of the genes.

2. A kit for detection of the lukS-PV and/or lukF-PV genes of a Staphylococcus in a sample, the kit comprising one or more capture probe(s) and one or more detector probe(s), wherein the capture probe(s) hybridize to a segment of the lukS-PV and/or lukF-PV genes and the detector probe(s) hybridize to a segment of the lukS-PV and/or lukF-PV genes.

3. A composition for detection of the lukS-PV and/or lukF-PV genes of a Staphylococcus, said composition comprising:
a) a probe comprising a nucleobase sequence selected from the group consisting of:
i) SEQ ID NO:X;
ii) a nucleobase sequence having at least 75% sequence identity to SEQ ID NO:X;
iii) a contiguous segment of i) at least 8 nucleobases long; and
iv) a contiguous segment of ii) at least 8 nucleobases long; and
b) a probe comprising a nucleobase sequence selected from the group consisting of:
i) SEQ ID NO:Y;
ii) a nucleobase sequence having at least 75% sequence identity to SEQ ID NO:Y;
iii) a contiguous segment of i) at least 8 nucleobases long; and
iv) a contiguous segment of ii) at least 8 nucleobases long;
wherein X is 1 and Y is 2; X is 1 and Y is 3; X is 1 and Y is 4; X is 1 and Y is 5; X is 2 and Y is 3; X is 2 and Y is 4; X is 2 and Y is 5; X is 3 and Y is 4; X is 3 and Y is 5; X is 4 and Y is 5; X is 9 and Y is 10; X is 9 and Y is 11; X is 9 and Y is 15; X is 9 and Y is 16; X is 10 and Y is 11; X is 10 and Y is 15; X is 10 and Y is 16; X is 11 and Y is 15; X is 11 and Y is 16; X is 15 and Y is 16; X is 21 and Y is 22; X is 21 and Y is 23; X is 4 and Y is 22; or X is 4 and Y is 23.

4. A method for detecting the tst gene of a Staphylococcus in a sample, the method comprising hybridizing DNA of the sample to one or more capture probe(s) and one or more detector probe(s), wherein the capture probe(s) hybridize to a segment of the tst gene, and the detector probe(s) hybridize to a segment of the tst gene, and detecting or not detecting hybridized detector probe(s), thereby determining the presence or absence, respectively, of the tst gene.

5. A kit for detection of the tst gene of a Staphylococcus in a sample, the kit comprising one or more capture probe(s) and one or more detector probe(s), wherein the capture probe(s) hybridize to a segment of the tst gene and the detector probe(s) hybridize to a segment of the tst gene.

6. A composition for detection of the tst gene of a Staphylococcus, said composition comprising:
a) a probe comprising a nucleobase sequence selected from the group consisting of:
i) SEQ ID NO:X;
ii) a nucleobase sequence having at least 75% sequence identity to SEQ ID NO:X;
iii) a contiguous segment of i) at least 8 nucleobases long; and
iv) a contiguous segment of ii) at least 8 nucleobases long; and
b) a probe comprising a nucleobase sequence selected from the group consisting of:
i) SEQ ID NO:Y;
ii) a nucleobase sequence having at least 75% sequence identity to SEQ ID NO:Y;
iii) a contiguous segment of i) at least 8 nucleobases long; and
iv) a contiguous segment of ii) at least 8 nucleobases long;
wherein X is 6 and Y is 7; X is 6 and Y is 8; X is 7 and Y is 8; X is 12 and Y is 13; X is 12 and Y is 14; or X is 13 and Y is 14.

7. A method for detecting the ileS-2 gene of a Staphylococcus in a sample, the method comprising hybridizing DNA of the sample to one or more capture probe(s) and one or more detector probe(s), wherein the capture probe(s) hybridize to a segment of the ileS-2 gene and the detector probe(s) hybridize to a segment of the ileS-2 gene, and detecting or not detecting hybridized detector probe(s), thereby determining the presence or absence, respectively, of the ileS-2 gene.

8. A kit for detection of the ileS-2 gene of a Staphylococcus in a sample, the kit comprising one or more capture probe(s) and one or more detector probe(s), wherein the capture probe(s) hybridize to a segment of the ileS-2 gene and the detector probe(s) hybridize to a segment of the ileS-2 gene.

9. A composition for detection of the ileS-2 gene of a Staphylococcus, said composition comprising:
a) a probe comprising a nucleobase sequence selected from the group consisting of:
i) SEQ ID NO:X;
ii) a nucleobase sequence having at least 75% sequence identity to SEQ ID NO:X;
iii) a contiguous segment of i) at least 8 nucleobases long; and
iv) a contiguous segment of ii) at least 8 nucleobases long; and
b) a probe comprising a nucleobase sequence selected from the group consisting of:
i) SEQ ID NO:Y;
ii) a nucleobase sequence having at least 75% sequence identity to SEQ ID NO:Y;
iii) a contiguous segment of i) at least 8 nucleobases long; and
iv) a contiguous segment of ii) at least 8 nucleobases long;
wherein X is 17 and Y is 18; or X is 19 and Y is 20.

10. A probe comprising a nucleic acid or a DNA mimic, wherein the nucleic acid or DNA mimic comprises:
a) SEQ ID NO:X;
b) a nucleobase sequence having at least 75% sequence identity to SEQ ID NO:X;
c) a contiguous segment of a) at least 8 nucleobases long; or
d) a contiguous segment of b) at least 8 nucleobases long;
wherein SEQ ID NO:X is a nucleobase sequence selected from the group consisting of SEQ ID NOs 1-23.

11. An array comprising probes attached to a support, wherein at least one probe comprises a nucleobase sequence selected from the group consisting of:
a) SEQ ID NO:X;
b) a nucleobase sequence having at least 75% sequence identity to SEQ ID NO:X;
c) a contiguous segment of a) at least 8 nucleobases long; and
d) a contiguous segment of b) at least 8 nucleobases long;
wherein SEQ ID NO:X is selected from SEQ ID NOs 1-23.

12. A method of determining the presence or absence of each of a combination of genes of a Staphylococcus, without amplifying DNA of the Staphylococcus, the method comprising:
a) for each of the genes, hybridizing DNA of the Staphylococcus to one or more capture probes attached to a support, wherein the capture probes hybridize to DNA comprising a segment of the gene, thereby producing captured DNA comprising a segment of the gene, if DNA comprising a segment of the gene is present;
b) for each of the genes, hybridizing one or more detector probes to the DNA, wherein the detector probes hybridize to DNA comprising a segment of the gene, thereby producing hybridized detector probes, if DNA comprising a segment of the gene is present; and
c) for each of the genes, detecting or not detecting each of the hybridized detector probes on the support, thereby determining the presence or absence, respectively, of each of the genes.


SEQUENCE LISTING (23 sequences)

SEQ ID NO: 1 - DNA, 40 bp, Staphylococcus aureus
aacgtatggcagaaatatggatgttactcatgctactaga

SEQ ID NO: 2 - DNA, 40 bp, Staphylococcus aureus
agtgaactggaaaactcatgaaattaaagtgaaaggacat

SEQ ID NO: 3 - DNA, 40 bp, Staphylococcus aureus
gcaacatcagattccgataagttaaaaatttctcagattt

SEQ ID NO: 4 - DNA, 40 bp, Staphylococcus aureus
aaagttatgataaagatacattaatactcaaagctgctgg

SEQ ID NO: 5 - DNA, 40 bp, Staphylococcus aureus
tacaaagccaaatccaaaagacactattagttctcaattt

SEQ ID NO: 6 - DNA, 38 bp, Staphylococcus aureus
gctacagattttacccctgttcccttatcatctaatca

SEQ ID NO: 7 - DNA, 40 bp, Staphylococcus aureus
gcatctacaaacgataatataaaggatttgctagactggt

SEQ ID NO: 8 - DNA, 40 bp, Staphylococcus aureus
tagtgaagttttagataattccttaggatctatgcgtata

SEQ ID NO: 9 - DNA, 40 bp, Staphylococcus aureus
aaatctgagaaatttttaacttatcggaatctgatgttgc

SEQ ID NO: 10 - DNA, 40 bp, Staphylococcus aureus
ccagcagctttgagtattaatgtatctttatcataacttt

SEQ ID NO: 11 - DNA, 40 bp, Staphylococcus aureus
aaattgagaactaatagtgtcttttggatttggctttgta

SEQ ID NO: 12 - DNA, 38 bp, Staphylococcus aureus
tgattagatgataagggaacaggggtaaaatctgtagc

SEQ ID NO: 13 - DNA, 40 bp, Staphylococcus aureus
accagtctagcaaatcctttatattatcgtttgtagatgc

SEQ ID NO: 14 - DNA, 40 bp, Staphylococcus aureus
tatacgcatagatcctaaggaattatctaaaacttcacta

SEQ ID NO: 15 - DNA, 40 bp, Staphylococcus aureus
tctagtagcatgagtaacatccatatttctgccatacgtt

SEQ ID NO: 16 - DNA, 40 bp, Staphylococcus aureus
atgtcctttcactttaatttcatgagttttccagttcact

SEQ ID NO: 17 - DNA, 39 bp, Staphylococcus aureus
ctcattgttggagatgtggtaatcctttgatatattatg

SEQ ID NO: 18 - DNA, 40 bp, Staphylococcus aureus
caataataataatatagagtggtttccttctcatattaag

SEQ ID NO: 19 - DNA, 39 bp, Staphylococcus aureus
cataatatatcaaaggattaccacatctccaacaatgag

SEQ ID NO: 20 - DNA, 40 bp, Staphylococcus aureus
cttaatatgagaaggaaaccactctatattattattattg

SEQ ID NO: 21 - DNA, 32 bp, Staphylococcus aureus
gataaagatacattaatactcaaagctgctgg

SEQ ID NO: 22 - DNA, 33 bp, Staphylococcus aureus
caaagccaaatccaaaagacactattagttctc

SEQ ID NO: 23 - DNA, 30 bp, Staphylococcus aureus
cagattctaatgactcagtaaacgttgtag